(19) United States
(12) Patent Application Publication
(10) Pub. No.: US 2009/0035365 A1
(43) Pub. Date: Feb. 5, 2009

(54) Density Controlled Capsule Particles and Methods of Making the Same

(76) Inventors: Lewis Michael Popplewell, Morganville, NJ (US); Yabin Lei, Holmdel, NJ (US)

Correspondence Address: INTERNATIONAL FLAVORS & FRAGRANCES INC., 521 WEST 57TH ST, NEW YORK, NY, 10019, US

(21) Appl. No.: 11/830,367

(22) Filed: Jul. 30, 2007

(51) Int. Cl. A61K 9/48; A61K 8/11; C08L 61/22; C08L 89/00; C09K 3/00

(52) U.S. Cl. 424/451; 252/182.11; 424/59; 424/65; 424/70.1; 524/498; 524/555; 524/597

(57) ABSTRACT

The present invention discloses density controlled capsule particle and methods for making the same for use in a wide range of consumer and personal care products.

### FIELD OF THE INVENTION

The present invention discloses densified capsule particles and methods for making the same for use in a wide range of consumer and personal care products.

### BACKGROUND OF INVENTION

Encapsulation of active material, such as fragrances, is well known in the art. Encapsulation provides advantages to the fragrance product including the protection of the fragrance in the capsule core by a shell until the fragrance is intended to be delivered. In particular, capsules are often designed to deliver their contents at a desired time by the capsule shell being compromised at the desired time.

Various encapsulation techniques have been explored for the delivery of fragrance molecules. In many end applications, the encapsulation of aroma chemicals is a necessary, sometimes, mandatory process to protect the active ingredient from the harsh application environments they are in. It can also prevent the loss and premature release of active ingredients in the targeted application. The encapsulation of aroma chemicals can enable one to control and modulate the release profiles of aroma chemicals to achieve the desired perfumery effects and consumer benefits.

In practical applications, the encapsulated product can be manufactured either as a solid or liquid slurry, or gel depending on the process and application. These encapsulated and concentrated systems can then be diluted and mixed into the final products. Since most of the fragrance oil has density of less than unity, the fragrance containing capsules will typically has an overall density of less than one. This can be quite problematic in practical applications. If the product is a liquid or slurry and the continuous phase is water, the light encapsulate can cream to the top of the sample container to promote coalescence and induce the formation a more concentrated layer that can be difficult to break and use. This can have a significant impact on the rheology of the system. Most importantly, it can lead to the uneven distribution of active ingredient.

The same problem exits, to a less degree, when the encapsulated material is diluted into the application medium. Upward creaming is normally observed when the encapsulate is dispersed into aqueous medium. Normally, these problems are addressed by adjusting the rheology of the capsule slurry or application medium. This in turn can lead to other product and performance issues such as undesirable thickening.

Encapsulation can greatly facilitate and enhance the application of fragrances. For example, when an encapsulated fragrance product is diluted into a fabric softener solution, long lasting perfumery effects can be achieved.

Key performance criteria of fragrance capsules used in these type of laundry applications are deposition (i.e. how many of the capsules actually remain on the substrate) and fragrance release (i.e. release of the payload in an appropriate manner). Optimizing capsule and product system performance against these performance criteria is a continuing challenge.

Inorganic particulates have been explored as delivery vehicles for active ingredients including fragrance. The majority of the systems are matrix based controlled release system. The use of various form of silica as carriers for a variety of active ingredients has been well documented because of its larger surface area and higher absorption capacity. For example, Syloid® 244, a form of silica, is commonly used as an oils absorbent for flavor encapsulation. The encapsulated systems are typically prepared by physical adsorption for liquid active and physical blending for solid ingredient. The finished product is generally present in the solid form and applied as such and release of the active ingredient occurs by passive diffusion. Because of their particulate and interfacial properties, these systems are often not compatible with liquid application media. Direct addition of these systems to a liquid product base often leads to release of the liquid actives and/or agglomeration in the product base. The finished product generally has poor storage stability and inferior performance.

Very little is known and disclosed regarding the encapsulation of the inorganic particulate materials as core materials in the core/shell type of encapsulates. This can be partially attributed to technical difficulties in creating a true core/shell structure involving this type of material. Self-assembled monolayer structures have been built around inorganic particulate, but they are not true core/shell capsules. A carefully engineered encapsulate containing these particulate materials can significantly expand their application.

### SUMMARY OF THE INVENTION

An object of the present invention is to provide densified capsule particles that have improved physical and storage stability, improved deposition, better rheological and mixing properties, and efficient release profile leading to significant perfumery benefits.

In one embodiment, a densified capsule particle is provided comprising a combination of an active ingredient, a densifying agent resident largely in the core and an encapsulating polymeric material.

In a further embodiment, a process for preparing a densified capsule particle is provided.

In yet another embodiment, the densified capsule particle can be used in range of consumer and personal care applications including rinse conditioner, cleaners, personal care, oral care, hair care, liquid body wash, sun screen formulations, liquid makeup, and textile adjuncts.

These and other objects and advantages of the present invention have been achieved by the encapsulation of core compositions created by the synergistic use of inorganic particulates and other densifying agents, fragrance oils, UV filters and pigments.

### DETAILED DESCRIPTION OF THE INVENTION

The terms densifying agents and densification agents may be used interchangeably throughout the specification.

In this invention, we describe a densified capsule particles and methods to make the same through the novel use of densifying agents and perfumery materials that can function as core materials and be encapsulated to produce multifunctional delivery systems. With the proper selection of core materials such as densifying agent and fragrance, the encapsulated system can have improved physical stability, rheological profile and mixing properties, as well as improved deposition and release leading to significant consumer benefits.

According to one embodiment of the invention, the densifying agents may be metal oxides selected from but not limited to titanium dioxide (TiO₂), zinc oxide (ZnO), Fe₂O₃, CO₂O₃, CoO, NiO, AgO, CuO, Zicornium (ZrO₂), silica, and other metal oxides. They should have specific density of greater than unity. Oxides that can function both as densification agent and provide additional functional properties are preferred. For example, titanium dioxide, Uvinul® TiO₂, can function as both a densification agent and UV filter; it is therefore a preferred material.

In a preferred embodiment the density of the densifying agent is greater than 1. It has been found by adding densifying agents to the core allows density to be independently adjusted and increased to an application optimum. In addition, hydrophobically modified metal oxides are preferred. Examples of metal oxides include, but are not limited to, Uvinul® TiO2, Z-COTE® HP1, T-lite™ SF. T-lite™ SF-S, T-lite™ MAX, and Z-COTE® MAX manufactured by BASF; Aerosil® R812, Aerosil® R972/R94 from Degussa; and Ti-Pure® R-700, and Ti-Select™ TS-6200 from Dupont.

The densifying agents may also be selected from organic compounds including brominated vegetable oil (BVO) and sucrose acetate isobutyrate. These densifying agents are available from Eastman chemical (Kingsport, Tenn. 37662) under the trade name: Sustane SAIB, Sustane SAIB MCT, Sustane SAIB ET-10, Eastman SAIB-100, Eastman SAIB-90EA, and Eastman SAIB-90. For the purpose of densification, any substances that possesses a density of greater than 1 and does not significantly react with the fragrance may be used. Furthermore, a material that is odorless or does not interfere with the primary odor of the fragrance is highly preferred. The selection can be made based on the chemical and physical compatibility of the densification agent and that of the fragrance core.

The densification agents may also be selected from inert metallic particles or metallic compounds or metallic alloys since these materials normally posses density of greater than 1.0 and can be highly effective in providing the desired density. Examples are silver (Ag), zinc (Zn), iron (Fe), cobalt (Co), Nickel (Ni), and copper (Cu). The preferred materials will be those that are compatible with the fragrance core.

In the case of a solid densification agent, the material can be of any physical dimension and morphology compatible with the desired capsule characteristics (e.g. size). The core materials can be selected from materials with dimensions ranging from a few nanometers to microns. As far as the physical dimension is concerned, the upper and lower limit of the core densification agent will be ultimately determined by the physical dimension of the capsules. For example, if one is to prepare a 30 micron densified capsule, the maximum physical dimension of the densification agent is limited to 30 micron or less. One can further speculate that, for optimal performance, there might exist a relationship between the physical dimension of the capsule and that of the core densification agent. For example, a larger capsule may need a densification agent with a larger physical size for better breakage and release. This may be explainable if the capsules breakage is by protrusion force. Likewise, a smaller capsule may benefit from material with a smaller grain size.

The core materials may further be hollow, porous, meso-porous, nano-porous or completed filled. The core materials can also be of any regular or irregular shape including sphere, square, needles, fibers, and ellipsoids. The physical dimension of the core materials can range from nanoscaled to micro-sized material. Preferably, as long the core materials can be encapsulated in the core of the capsule and the fragrance core will remain as liquid after the fragrance core is mixed with the densification agent.

It is commonly known that the apparent or observed performance (P) of encapsulated product is likely dependent on a matrix of variables including fragrance capsule stability which measures the amount of fragrance retained in application and after extended storage, capsule deposition, and capsules breakage and fragrance release. This can be formulated as:

Performance(P)=Fₙ(stability, deposition, release)

The improved performance of densified capsules, compared with the non-densified capsules, can occur via better stability, or better deposition, or more efficient breakage or release or combination of all the above factors. Thus, the performance of the capsules is related to the density of the fragrance capsules:

Performance(P)=Fₙ(density of the capsule)

Since the fragrance capsules is composed of three components, the shell wall, the fragrance core and the densification agent, the density of the capsule can be calculated using the following equation:

$${ρ}_{c} = \frac{{ρ}_{s} {ρ}_{f} {ρ}_{d} ( {W}_{s} + {W}_{f} + {W}_{d} )}{( {ρ}_{f} {ρ}_{d} {W}_{s} + {ρ}_{s} {ρ}_{d} {W}_{f} + {ρ}_{s} {ρ}_{f} {W}_{d} )}$$

### Where:

ρ_{c}: over all density of capsules

Wₛ and ρₛ: weight and density of shell material

W_{f} and ρ_{f}: weight and density of the fragrance oil

W_{d} and ρ_{d}: weight and density of densification agent

It is quite apparent that the density of the capsule can be manipulated by the proper choice of capsule wall, fragrance oil, and densification agent. For a given polymer wall material, the density of the capsule can adjusted by the selection of appropriate fragrance oil and densification agents. In practice, one can correlate the performance of an encapsulated product by making a series of capsules of varying density to optimize the performance of a particular product in targeted application. The majority of the densification agent is expected to be occluded in the core, although its diffusion to the wall region can occur in the process. For example, as size declines, density increases due to the fact that there is more wall per capsule. Some of the best results indicate that small size and higher density yield the best performance.

It is preferable that metal oxides such as titanium dioxide and zinc oxides are used in the core as they are often incorporated into cosmetic product to provide protection for the skin and hair from UV radiation. Both the rutile and anatase forms of titanium dioxide can be used in the invention. Additionally, the materials can be coated with hydrophobic materials such as trimethoxycaprylsilane, triethoxycaprylsilane, diphenyl capryl methicone, dimethoxydiphenylsilane/triethoxycaprylsilane crosspolymer. The particle size of the material can vary from a few nanometers to a few hundred micrometers. The physical dimension of the densification agent will be determined by the physical size of the capsules.

Other UV filters may optionally be incorporated when metal oxides other than titanium dioxide and zinc oxides are used. These UV filters can be selected from benzophenones, diphyenyl cyanoacrylate, cinnamates, octyltroazone, methylbenzylidene camphor, p-aminobenzoic acid derivatives or dibenzoyl methane derivatives. For optimal performance, these organic UV filters can be synergistically used with titanium dioxide and zinc dioxide. When the organic filters are used alone, the materials having the highest specific density are preferred used.

The fragrance or flavor materials to be encapsulated in the core can be selected from a wide range of commercial and proprietary products. Any number of ingredients in the fragrance system can be used as long as the formulations give rise to desired sensory profile and benefits. Single perfumery components can also be used, as are diluents and solvents.

The encapsulation process is a very critical step in the current invention. Without encapsulation, fragrance load metal oxides such as titanium dioxides have limited direct applications as the materials are highly hydrophobic and incompatible with aqueous solution in which many of the products are formulated. The process in the current invention makes this possible because the surface properties of the oxide are beneficially modified via the encapsulation process, but not by the traditional surface coating process. The wall polymer of the capsule provides a bridge between the oxides and application mediums. This allows the realization of the many potential benefits of the densification agents. This benefit is manifested by the improved consumer and sensory performance that can occur through reduced leakage of the core materials, enhanced deposition, better breakage and release.

The encapsulated systems can be prepared by several processes. In one process, the metal oxide such as titanium dioxide is pre-blended with the fragrance oil under slight shear to form a homogenous dispersion. This mixture can then used be as a multifunctional core. The coating polymer is pre-formed with suitable monomers. The core material is then sheared into an aqueous solution of the coating polymer at the desired concentration. The polymerization reaction is allowed to proceed further under constant agitation at elevated temperature. The polymerization reaction is stopped after the reaction or encapsulation is complete. Encapsulation of the core materials can be monitored by optical microscope. The collected slurry can be used in many applications.

In another process, the core composition can be directly blended into the polymer precursor solution under shearing. The polymerization reaction is allowed to proceed further under constant agitation at elevated temperature. The polymerization reaction is stopped after the reaction is complete. The progress of reaction and encapsulation of the core materials can be monitored by optical microscope. The collected slurry can be used in any direct application.

Multi-component and multi-functional encapsulate systems with different physical and performance properties can be prepared by the current invention. In a preferred embodiment, one of the benefits of the invention is that it greatly improves the perfumery and sensory benefits through reduced fragrance leaching, enhanced deposition and efficient release and breakage.

In addition, when UV filtering pigments such as titanium dioxide or zinc oxide are used as part of core, the encapsulated materials have several unique attributes. The high specific density of TiO₂ and its excellent miscibility with fragrance oils allows one to adjust the density of core material in such way that the fragrance capsules will have a density that is optimally matched with the density of the dispersed phase such as water. As a result, the capsules will distribute homogenously in the slurry for uniform dosing. The rheological property of the slurry is also dramatically improved since there will be no creaming occurring. This can greatly facilitate the application of the product.

Because pigments such as titanium dioxide and zinc dioxide are UV filters, they can protect other core ingredients such fragrance and flavor from UV radiation. When the encapsulated product is used in personal care products such sun screen lotions and hair care products, they can provide protection for the skin and hair. At the same time, they can delivery the long lasting perfumery benefits. When the encapsulated product is used in fabric care and textile, deposited TiO₂ can provide further protection for the substrate from harmful UV rays.

The encapsulated fragrance can also be easily incorporated into liquid makeup and color cosmetics formulations and products. Currently, micro- and nano-grain sized titanium dioxide is being used in some of the products by direct emulsification into the application medium and the process can be problematic. The fragrance is added as a separated ingredient. The direct addition of encapsulate materials containing both oxide such as titanium dioxide and fragrance can greatly simplify the manufacturing process to provide the needed pigmentation ingredients and long lasting perfumery effects. The use of iron oxide and fragrance in the core will allow the products easily incorporated into color cosmetics.

Fragrance capsules containing particulates such as TiO₂ have higher specific density and UV protection properties in addition to its perfumery benefits. These properties can be advantageously used in textile products. The density of the capsule can be tuned to allow facile mixing in the textile bathing and finishing processes when upward creaming can cause problems. The deposited TiO₂ containing capsule can afford the light fastness for the fabrics which are dyed with organic dyes and pigments as it can prevent the substrates from photo-degradation. It can further minimize the exposure of human skin from harmful UV radiation. Simultaneously, it can deliver the perfumery benefits of the product.

One can also use a mixture of inorganic oxides such as silica and TiO₂. The use of mixture of metal oxides in the core provides means for the systematic variation of the density of the core so that optimal core composition can be used. This will lead to better rheology and stability for the encapsulated product. Since silica is not a UV filter, organic and inorganic UV filers may be used in conjunction with it to produce the multi-functional delivery systems.

Organic UV filtering material can also be used in the current invention. For efficiency and better stability, material with higher specific density is preferred. However, as long as the density of the core mixture is great than one, any organic filters and combinations of such materials may be used.

To engineer encapsulate with the maximum UV protection, the inorganic UV filter pigment can be incorporated into core with the organic UV filters because of they complement each other in light absorption and UV protection. Optimal system can be formulation by the careful study of the UV filtering profile of the system.

The inclusion of densifying agents in the current invention offers one the possibility to engineer the release profile of the fragrance ingredient from the capsules. In encapsulated fragrance product, the release of fragrance and flavors can occur either by physical diffusion or mechanical disruption. The inclusion of abrasive metal oxides can induce capsule breakage when they are deposited onto the substrates and are perturbed with external forces. It can also promote the self-diffusion of fragrance molecules. All these factors can lead to more desirable release profiles and superior performance.

The current invention will enable the facile and precise control of the wall thickness of the capsules by adjusting the concentration of the polymer solution, and thus the amount of capsule wall. Physical properties of the capsules can be tuned accordingly.

Fragrance capsules prepared by the current application can be readily incorporated into consumer products such as rinse conditioner, liquid and powder detergent, cleaners, personal care, hair care, sun screen formulation, liquid makeup, and textile.

The benefits of fragrance capsule can be demonstrated by perfumery benefits they bring into consumer products. For examples, to evaluate their benefits in rinse conditioner (RC) or liquid detergent, the fragrance capsule can be blended into the base product. The product can then be used to conduct a laundering experiment using commercially available towels. The efficacy of a consumer product containing capsules can be measured by the perfumery intensity before any physical force is applied to the fabric, the pre-rubbing intensity (Iₚᵣₑ); and when the fabric is subject to some sort of mechanical perturbation such as physical rubbing, the post-rubbing intensity (Iₚₒₛₜ); and the ratio of (Iₚₒₛₜ)/(Iₚᵣₑ). As it is common for consumers or fragrance users to expect good and strong “smell” when they are in contact with a perfumed object, it is very desirable for an encapsulated fragrance to generate as high a pre-rubbing intensity as possible and to have an optimal overall intensity.

We have surprisingly discovered that the densified fragrance capsules prepared from the current invention has significantly increased the Iₚᵣₑ intensity in the consumer products containing these capsules while maintaining the high overall fragrance intensity. The finished product that has a more favorable release profile and can delivery the perfumery benefits at all stages of application. Such phenomena have not been revealed in the public domain before.

One factor that has contributed to the improved performance is the greatly improved of deposition in the densified capsules. The discovery is quite significant since deposition is a critical factor that will determine the performance of consumer product containing fragrance capsule in a particular end application. Optimal and enhanced deposition is a prerequisite for excellent overall performance. Because of the many steps involved in the application of the encapsulated product, the development of an encapsulated system with enhanced deposition is a challenging task. Normally a deposition polymer is used in most consumer products. The excellent deposition we obtained, in the absence of a deposition polymer, is very significant and represents a major technological breakthrough. Because of the multiple steps involved in fragrance capsule deposition, understanding the exact mechanism by which the enhanced deposition is materialized requires far more than a straight forward analysis.

Encapsulation of active material such as fragrances is known in the art, see for example U.S. Pat. Nos. 2,800,457, 3,870,542, 3,516,941, 3,415,758, 3,041,288, 5,112,688, 6,329,057, and 6,261,483. Another discussion of fragrance encapsulation is found in the Kirk-Othmer Encyclopedia.

The encapsulating polymers include those formed from, acrylates, acrylamide, acrylate-co-acrylamide, melamine-formaldehyde or urea-formaldehyde condensates, as well as similar types of aminoplasts. The encapsulating material or the wall forming materials can be selected from a wide range of polymers, co-polymers, cross-linked polymers including melamine formaldehyde resins, polyureathane polymers and resins. The polymer and copolymers can also be selected from polyacrylates, polyacrylamides, poly(acrylamide-co-acrylate), polyvinyl alcohol, poly(ethylene-co-vinyl acetate) (EVA), poly(vinylpyrrolidone-co-styrene), poly(ethylene oxide-co-propylene oxide), poly(styrene-co-maleic anhydride), poly(ethylene-alt-maleic anhydride) (EMA), and salt of poly(methacryloyloxyethyl).

Other wall forming materials include polyurethane, polysiloxanes, polyurea, polyamide, polyimide, polyvinyl alcohol, polyanhydride, polyolefin, polysulfone, polysaccaharide, protein, polylactide (PLA), polyglycolide (PGA), polyorthoester, polyphosphazene, silicone, lipid, modified cellulose, gums, polystyrene, and polyesters or combinations of these materials. Other polymeric materials that are functional are ethylene maleic anyhydride copolymer, styrene maleic anyhydride copolymer, ethylene vinyl acetate copolymer and lactide glycolide copolymer. Bio-polymers that are derived from alginate, chitosan, collegen, dextran, gelatin, and starch can also be used as the encapsulating materials. Additionally, microcapsules made via the simple or complex coacervation of gelatin are also preferred for use with the coating.

The ratio of wall forming polymer to that of core by weight independent of the slurry composition falls in the range of about 1:100 to about 50:1, more preferable in the range of about 1:50 to about 25:1, even more preferable in the range of about 1:25 to about 10:1 and most preferably of about 1:10 to about 5:1.

A representative process used for aminoplast encapsulation is disclosed in U.S. Pat. No. 3,516,941 though it is recognized that many variations with regard to material and process steps are possible. A representative process used for gelatin encapsulation is disclosed in U.S. Pat. No. 2,800,457 though it is recognized that many variations with regard to material and process steps are possible. Both of these processes are discussed in the context of fragrance encapsulation for use in consumer products in U.S. Pat. Nos. 4,145,184 and 5,112,688 respectively.

Well known material such as solvents, surfactants, emulsifiers, and the like can be used in addition to the polymers described throughout the invention to encapsulate the active material such as fragrance without departing from the scope of the present invention. It is understood that the term encapsulated is meant to mean that the active material is substantially covered in its entirety. Encapsulation can provide pore vacancies or interstitial openings depending on the encapsulation techniques employed. More preferably the entire active material portion of the present invention is encapsulated.

Fragrance capsules known in the art consists of a core of various ratios of fragrance and solvent material, a wall or shell comprising a three-dimensional cross-linked network of an aminoplast resin, more specifically a substituted or un-substituted acrylic acid polymer or co-polymer cross-linked with a urea-formaldehyde pre-condensate or a melamine-formaldehyde pre-condensate.

Microcapsule formation using mechanisms similar to the foregoing mechanism, using (i) melamine-formaldehyde or urea-formaldehyde pre-condensates and (ii) polymers containing substituted vinyl monomeric units having proton-donating functional group moieties (e.g. sulfonic acid groups or carboxylic acid anhydride groups) bonded thereto is disclosed in U.S. Pat. No. 4,406,816 (2-acrylamido-2-methyl-propane sulfonic acid groups), UK published Patent Application GB 2,062,570 A (styrene sulfonic acid groups) and UK published Patent Application GB 2,006,709 A (carboxylic acid anhydride groups).

The cross-linkable acrylic acid polymer or co-polymer microcapsule shell wall precursor has a plurality of carboxylic acid moieties, to with:

The cross-linkable acrylic acid polymer or co-polymer microcapsule shell wall precursor has a plurality of carboxylic acid moieties, to with:

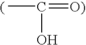

and is preferably one or a blend of the following:
(i) an acrylic acid polymer;
(ii) a methacrylic acid polymer;
(iii) an acrylic acid-methacrylic acid co-polymer;
(iv) an acrylamide-acrylic acid co-polymer;
(v) a methacrylamide-acrylic acid co-polymer;
(vi) an acrylamide-methacrylic acid co-polymer;
(vii) a methacrylamide-methacrylic acid co-polymer;
(viii) a C₁-C₄ alkyl acrylate-acrylic acid co-polymer;
(ix) a C₁-C₄ alkyl acrylate-methacrylic acid co-polymer;
(x) a C₁-C₄ alkyl methacrylate-acrylic acid co-polymer;
(xi) a C₁-C₄ alkyl methacrylate-methacrylic acid co-polymer;
(xii) a C₁-C₄ alkyl acrylate-acrylic acid-acrylamide co-polymer;
(xiii) a C₁-C₄ alkyl acrylate-methacrylic acid-acrylamide co-polymer;
(xiv) a C₁-C₄ alkyl methacrylate-acrylic acid-acrylamide co-polymer;
(xv) a C₁-C₄ alkyl methacrylate-methacrylic acid-acrylamide co-polymer;
(xvi) a C₁-C₄ alkyl acrylate-acrylic acid-methacrylamide co-polymer;
(xvii) a C₁-C₄ alkyl acrylate-methacrylic acid-methacrylamide co-polymer;
(xviii) a C₁-C₄ alkyl methacrylate-acrylic acid-methacrylamide co-polymer; and
(xix) a C₁-C₄ alkyl methacrylate-methacrylic acid-methacrylamide co-polymer;
   and more preferably, an acrylic acid-acrylamide copolymer.

When substituted or un-substituted acrylic acid co-polymers are employed in the practice of our invention, in the case of using a co-polymer having two different monomeric units, e.g. acrylamide monomeric units and acrylic acid monomeric units, the mole ratio of the first monomeric unit to the second monomeric unit is in the range of from about 1:9 to about 9:1, preferably from about 3:7 to about 7:3. In the case of using a co-polymer having three different monomeric units, e.g. ethyl methacrylate, acrylic acid and acrylamide, the mole ratio of the first monomeric unit to the second monomeric unit to the third monomeric unit is in the range of 1:1:8 to about 8:8:1, preferably from about 3:3:7 to about 7:7:3.

The molecular weight range of the substituted or un-substituted acrylic acid polymers or co-polymers useful in the practice of our invention is from about 5,000 to about 1,000,000, preferably from about 10,000 to about 100,000. The substituted or un-substituted acrylic acid polymers or co-polymers useful in the practice of our invention may be branched, linear, star-shaped, dendritic-shaped or may be a block polymer or copolymer, or blends of any of the aforementioned polymers or copolymers.

Such substituted or un-substituted acrylic acid polymers or co-polymers may be prepared according to any processes known to those skilled in the art, for example, U.S. Pat. No. 6,545,084.

The urea-formaldehyde and melamine-formaldehyde pre-condensate microcapsule shell wall precursors are prepared by means of reacting urea or melamine with formaldehyde where the mole ratio of melamine or urea to formaldehyde is in the range of from about 10:1 to about 1:6, preferably from about 1:2 to about 1:5. For purposes of practicing our invention, the resulting material has a molecular weight in the range of from about 150 to about 3000. The resulting material may be used ‘as-is’ as a crosslinking agent for the aforementioned substituted or un-substituted acrylic acid polymer or copolymer or it may be further reacted with a C1-C6 alkanol, e.g. methanol, ethanol, 2-propanol, 3-propanol, 1-butanol, 1-pentanol or 1-hexanol, thereby forming a partial ether where the mole ratio of melamine or urea:formaldehyde:alkanol is in the range of 1:(0.1-6):(0.1-6). The resulting ether moiety-containing product may by used ‘as-is’ as a cross-linking agent for the aforementioned substituted or un-substituted acrylic acid polymer or copolymer, or it may be self-condensed to form dimers, trimers and/or tetramers which may also be used as cross-linking agents for the aforementioned substituted or un-substituted acrylic acid polymers or co-polymers. Methods for formation of such melamine-formaldehyde and urea-formaldehyde pre-condensates are set forth in U.S. Pat. No. 3,516,846, U.S. Pat. No. 6,261,483, and Lee et al. J. Microencapsulation, 2002, Vol. 19, No. 5, pp 559-569, “Microencapsulation of fragrant oil via in situ polymerization: effects of pH and melamine-formaldehyde molar ratio”. Examples of urea-formaldehyde pre-condensates useful in the practice of our invention are URAC 180 and URAC 186, trademarks of Cytec Technology Corp. of Wilmington, Del. 19801, U.S.A. Examples of melamine-formaldehyde pre-condensates useful in the practice of our invention are CYMEL U-60, CYMEL U-64 and CYMEL U-65, trademarks of Cytec Technology Corp. of Wilmington, Del. 19801, U.S.A. In the practice of our invention it is preferable to use as the precondensate for cross-linking the substituted or un-substituted acrylic acid polymer or co-polymer. The melamine-formaldehyde pre-condensate having the structure:

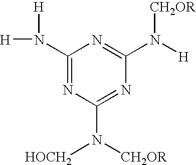

wherein each of the R groups are the same or different and each represents hydrogen or C1-C6 lower alkyl, e.g. methyl, ethyl, 1-propyl, 2-propyl, 1-butyl, 2-butyl, 2-methyl-1-propyl, 1-pentyl, 1-hexyl and/or 3-methyl-1-pentyl.

The range of mole ratios of urea-formaldehyde precondensate or melamine-formaldehyde pre-condensate: substituted or un-substituted acrylic acid polymer or co-polymer is in the range of from about 9:1 to about 1:9, preferably from about 5:1 to about 1:5 and most preferably from about 2:1 to about 1:2.

In another embodiment of the invention, microcapsules with polymer(s) comprising primary and/or secondary amine reactive groups or mixtures thereof and crosslinkers as disclosed in commonly assigned U.S. patent application Ser. No. 11/123,898.

The amine polymers can possess primary and/or secondary amine functionalities and can be of either natural or synthetic origin. Amine containing polymers of natural origin are typically proteins such as gelatin and albumen, as well as some polysaccharides. Synthetic amine polymers include various degrees of hydrolyzed polyvinyl formamides, polyvinylamines, polyallyl amines and other synthetic polymers with primary and secondary amine pendants. Examples of suitable amine polymers are the Lupamin series of polyvinyl formamides (available from BASF). The molecular weights of these materials can range from 10,000 to 1,000,000.

The polymers containing primary and/or secondary amines can be used with any of the following comonomers in any combination:

1. Vinyl and acrylic monomers with:

a. alkyl, aryl and silyl substituents;

b. OH, COOH, SH, aldehyde, trimonium, sulfonate, NH2, NHR substiuents;

c. vinyl pyridine, vinyl pyridine-N-oxide, vinyl pyrrolidon

2. Cationic monomers such as dialkyl dimethylammonium chloride, vinyl imidazolinium halides, methylated vinyl pyridine, cationic acrylamides and guanidine-based monomers

3. N-vinyl formamide and any mixtures thereof. The ratio amine monomer/total monomer ranges from 0.01-0.99, more preferred from 0.1-0.9.

The following represents a general formula for the amine-containing polymer material:

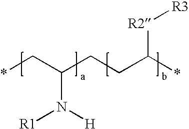

wherein R is a saturated or unsaturated alkane, dialkylsiloxy, dialkyloxy, aryl, alkylated aryl, and that may further contain a cyano, OH, COOH, NH2, NHR, sulfonate, sulphate, —NH2, quaternized amines, thiols, aldehyde, alkoxy, pyrrolidone, pyridine, imidazol, imidazolinium halide, guanidine, phosphate, monosaccharide, oligo or polysaccharide.

R1 is H, CH3, (C═O)H, alkylene, alkylene with unsaturated C—C bonds, CH2-CROH, (C═O)—NH—R, (C═O)—(CH2)n—OH, (C═O)—R, (CH₂)n-E, —(CH2-CH(C═O))n—XR, —(CH2)n-COOH, —(CH2)n-NH2, —CH2)n-(C═O)NH2, E is an electrophilic group; wherein a and b are integers or average numbers (real numbers) from about 100-25,000.

R2 can be nonexistent or the functional group selected from the group consisting of —COO—, —(C═O)—, —O—, —S—, —NH—(C═O)—, —NR1-, dialkylsiloxy, dialkyloxy, phenylene, naphthalene, alkyleneoxy. R3 can be the same or selected from the same group as R1.

Additional copolymers with amine monomers are provided having the structure:

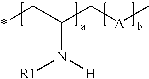

R1 is H, CH3, (C═O)H, alkylene, alkylene with unsaturated C—C bonds, CH2-CROH, (C═O)—NH—R, (C═O)—(CH2)n—OH, (C═O)—R, (CH2)n-E, —(CH2-CH(C═O))n—XR, —(CH2)n—COOH, —(CH2)n—NH2, —CH2)n—(C═O)NH2, E is an electrophilic group; wherein a and b are integers or average numbers (real numbers) from about 100-25,000; wherein R is a saturated or unsaturated alkane, dialkylsiloxy, dialkyloxy, aryl, alkylated aryl, and that may further contain a cyano, OH, COOH, NH2, NHR, sulfonate, sulphate, —NH2, quaternized amines, thiols, aldehyde, alkoxy, pyrrolidone, pyridine, imidazol, imidazolinium halide, guanidine, phosphate, monosaccharide, oligo or polysaccharide.

The comonomer, represented by A, can contain an amine monomer and a cyclic monomer wherein A can be selected from the group consisting of aminals, hydrolyzed or non-hydrolyzed maleic anhydride, vinyl pyrrolidine, vinyl pyridine, vinyl pyridine-N-oxide, methylated vinyl pyridine, vinyl naphthalene, vinyl naphthalene-sulfonate and mixtures thereof.

When A is an aminal the following general structure can represent the aminal:

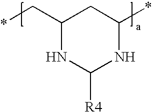

wherein R4 is selected from the group consisting of H, CH3, (C═O)H, alkylene, alkylene with unsaturated C—C bonds, CH2-CROH, (C═O)—NH—R, (C═O)—(CH2)n—OH, (C═O)—R, (CH2)n-E, —(CH2-CH(C═O))n—XR, —(CH2)n-COOH, —(CH2)n-NH2, —CH2)n-(C═O)NH2, E is an electrophilic group; wherein R is a saturated or unsaturated alkane, dialkylsiloxy, dialkyloxy, aryl, alkylated aryl, and that may further contain a cyano, OH, COOH, NH2, NHR, sulfonate, sulphate, —NH2, quaternized amines, thiols, aldehyde, alkoxy, pyrrolidone, pyridine, imidazol, imidazolinium halide, guanidine, phosphate, monosaccharide, oligo or polysaccharide.

In addition instead of amine-containing polymers it is possible to utilize amine-generating polymers that can generate primary and secondary amines during the microcapsule formation process as disclosed in commonly assigned U.S. patent application Ser. No. 11/123,898.

The crosslinkers can be selected from the group consisting of aminoplasts, aldehydes such as formaldehyde and acetaldehyde, dialdehydes such as glutaraldehyde, epoxy, active oxygen such as ozone and OH radicals, poly-substituted carboxylic acids and derivatives such as acid chlorides, anyhydrides, isocyanates, diketones, halide-substituted, sulfonyl chloride-based organics, inorganic crosslinkers such as Ca2+, organics capable of forming azo, azoxy and hydrazo bonds, lactones and lactams, thionyl chloride, phosgene, tannin/tannic acid, polyphenols and mixtures thereof. Furthermore, processes such as free radical and radiation crosslinking can be used according to the present invention. Examples of free radical initiators are benzoyl peroxide, sodium persulfate, azoisobutylnitrile (AIBN) and mixtures thereof.

With respect to the crosslinker, wall properties are influenced by two factors: the degree of crosslinking and the hydrophobic or hydrophilic nature of the crosslinker. The quantity and reactivity of the crosslinker determine the degree of crosslinking. The degree of crosslinking influences the microcapsule wall permeability by forming physical barriers towards diffusion. Walls made from crosslinkers possessing low-reactive groups will have smaller degrees of crosslinking than walls made from high-reactive crosslinkers. If a high degree of crosslinking is desired from a low-reactive crosslinker, more is added. If a low degree of crosslinking is desired from a high-reactive crosslinker then less is added. The nature and quantity of the crosslinker can also influence the hydrophobicity/hydrophilicity of the wall. Some crosslinkers are more hydrophobic than others and these can be used to impart hydrophobic qualities to the wall, with the degree of hydrophobicity directly proportional to the quantity of crosslinker used.

The degree of crosslinking and degree of hydrophobicity can result from a single crosslinker or a combination of crosslinkers. A crosslinker that is highly reactive and hydrophobic can be used to create microcapsule walls with a high degree of crosslinking and a hydrophobic nature. Single crosslinkers that possess both these qualities are limited and thus crosslinker blends can be employed to exploit these combinations. Crosslinkers possessing high reactivities but low hydrophobicities can be used in combination with a low reactive, high hydrophobicity crosslinker to yield walls with high degrees of crosslinking and high hydrophobicity. Suitable crosslinkers are disclosed in commonly assigned U.S. patent application Ser. No. 11/123,898.

(A) Copolymers containing primary and/or secondary amine. When amine-containing polymers are employed in the practice of the invention, in the case of using a co-polymer having two different monomeric units, e.g. Lupamin 9030 (copolymer of vinyl amine and vinyl formamide), the mole ratio of the first monomeric unit to the second monomeric unit is in the range of from about 0.1:0.9 to about 0.9:0.1, preferably from about 1:9 to about 9:1. In the case of using a co-polymer having three different monomeric units, e.g. a copolymer of vinyl amine, vinyl formamide and acrylic acid, the mole ratio of the reactive monomer (i.e. vinyl amine+acrylic acid) in the total polymer ranging from about 0.1 to about 0.9, more preferably from about 1 to about 9.

(B) Branched amine containing polymers such as ethylene imines (Lupasol series of BASF) and ethoxylated ethylene imines.

(C) Mixtures of amine containing polymers and other polymers that contain other reactive groups such as COOH, OH, and SH.

The molecular weight range of the substituted or un-substituted amine-containing polymers or co-polymers and mixtures thereof, useful in the practice of our invention is from about 1,000 to about 1,000,000, preferably from about 10,000 to about 500,000. The substituted or un-substituted amine-containing polymers or co-polymers useful in the practice of our invention may be branched, linear, star-shaped, graft, ladder, comb/brush, dendritic-shaped or may be a block polymer or copolymer, or blends of any of the aforementioned polymers or copolymers. Alternatively, these polymers may also possess thermotropic and/or lyotropic liquid crystalline properties.

Particle and microcapsule diameter can vary from about 1 micron to about 2000 microns, preferably from about 5 microns to about 500 microns and most preferably from about 10 to about 200 microns. The microcapsule distribution can be narrow, broad, or multi-modal. Each modal of the multi-modal distributions may be composed of different types of microcapsule chemistries.

Once the fragrance material is encapsulated a cationically charged water-soluble polymer may be applied to the fragrance encapsulated polymer. This water-soluble polymer can also be an amphoteric polymer with a ratio of cationic and anionic functionalities resulting in a net total charge of zero and positive, i.e., cationic. Those skilled in the art would appreciate that the charge of these polymers can be adjusted by changing the pH, depending on the product in which this technology is to be used. Any suitable method for coating the cationically charged material onto the encapsulated fragrance material can be used. The nature of suitable cationically charged polymers for assisted microcapsule delivery to interfaces depends on the compatibility with the microcapsule wall chemistry since there has to be some association to the microcapsule wall. This association can be through physical interactions, such as hydrogen bonding, ionic interactions, hydrophobic interactions, electron transfer interactions or, alternatively, the polymer coating could be chemically (covalently) grafted to the microcapsule or particle surface. Chemical modification of the microcapsule or particle surface is another way to optimize anchoring of the polymer coating to microcapsule or particle surface. Furthermore, the microcapsule and the polymer need to want to go to the desired interface and, therefore, need to be compatible with the chemistry (polarity, for instance) of that interface. Therefore, depending on which microcapsule chemistry and interface (e.g., cotton, polyester, hair, skin, wool) is used the cationic polymer can be selected from one or more polymers with an overall zero (amphoteric: mixture of cationic and anionic functional groups) or net positive charge, based on the following polymer backbones: polysaccharides, polypeptides, polycarbonates, polyesters, polyolefinic (vinyl, acrylic, acrylamide, poly diene), polyester, polyether, polyurethane, polyoxazoline, polyamine, silicone, polyphosphazine, olyaromatic, poly heterocyclic, or polyionene, with molecular weight (MW) ranging from about 1,000 to about 1000,000,000, preferably from about 5,000 to about 10,000,000. As used herein molecular weight is provided as weight average molecular weight. Optionally, these cationic polymers can be used in combination with nonionic and anionic polymers and surfactants, possibly through coacervate formation.

A more detailed list of cationic polymers that can be used to is provided below:

Polysaccharides include but are not limited to guar, alginates, starch, xanthan, chitosan, cellulose, dextrans, arabic gum, carrageenan, hyaluronates. These polysaccharides can be employed with:

(a) cationic modification and alkoxy-cationic modifications, such as cationic hydroxyethyl, cationic hydroxy propyl. For example, cationic reagents of choice are 3-chloro-2-hydroxypropyl trimethylammonium chloride or its epoxy version. Another example is graft-copolymers of polyDADMAC on cellulose like in Celquat L-200 (Polyquaternium-4), Polyquaternium-10 and Polyquaternium-24, commercially available from National Starch, Bridgewater, N.J.;

(b) aldehyde, carboxyl, succinate, acetate, alkyl, amide, sulfonate, ethoxy, propoxy, butoxy, and combinations of these functionalities. Any combination of Amylose and Mylopectin and overall molecular weight of the polysaccharide; and

(c) any hydrophobic modification (compared to the polarity of the polysaccharide backbone).

The above modifications described in (a), (b) and (c) can be in any ratio and the degree of functionalization up to complete substitution of all functionalizable groups, and as long as the theoretical net charge of the polymer is zero (mixture of cationic and anionic functional groups) or preferably positive. Furthermore, up to 5 different types of functional groups may be attached to the polysaccharides. Also, polymer graft chains may be differently modified than the backbone. The counterions can be any halide ion or organic counter ion. As disclosed in U.S. Pat. No. 6,297,203 and U.S. Pat. No. 6,200,554.

Another source of cationic polymers contain protonatable amine groups so that the overall net charge is zero (amphoteric: mixture of cationic and anionic functional groups) or positive. The pH during use will determine the overall net charge of the polymer. Examples are silk protein, zein, gelatin, keratin, collagen and any polypeptide, such as polylysine.

Further cationic polymers include poly vinyl polymers, with up to 5 different types of monomers, having the monomer generic formula —C(R2)(R1)-CR2R3-. Any co-monomer from the types listed in this specification may also be used. The overall polymer will have a net theoretical positive charge or equal to zero (mixture of cationic and anionic functional groups). Where R1 is any alkanes from C1-C25 or H; the number of double bonds ranges from 0-5. Furthermore, R1 can be an alkoxylated fatty alcohol with any alkoxy carbon-length, number of alkoxy groups and C1-C25 alkyl chain length. R1 can also be a liquid crystalline moiety that can render the polymer thermotropic liquid crystalline properties, or the alkanes selected can result in side-chain melting. In the above formula R2 is H or CH3; and R3 is —Cl, —NH2 (i.e., poly vinyl amine or its copolymers with N-vinyl formamide. These are sold under the name Lupamin 9095 by BASF Corporation), —NHR1, —NR1R2, —NR1R2R6 (where R6=R1, R2, or —CH2-COOH or its salt), —NH—C(O)—H, —C(O)—NH2 (amide), —C(O)—N(R2)(R2′)(R2″), —OH, styrene sulfonate, pyridine, pyridine-N-oxide, quaternized pyridine, imidazolinium halide, imidazolium halide, imidazol, piperidine, pyrrolidone, alkyl-substituted pyrrolidone, caprolactam or pyridine, phenyl-R4 or naphthalene-R5 where R4 and R5 are R1, R2, R3, sulfonic acid or its alkali salt —COOH, —COO— alkali salt, ethoxy sulphate or any other organic counter ion. Any mixture or these R3 groups may be used. Further suitable cationic polymers containing hydroxy alkyl vinyl amine units, as disclosed in U.S. Pat. No. 6,057,404.

Another class of material is polyacrylates, with up to 5 different types of monomers, having the monomer generic formula:

—CH(R1)-C(R2)(CO—R3-R4)-. Any co-monomer from the types listed in this specification may also be used. The overall polymer will have a net theoretical positive charge or equal to zero (mixture of cationic and anionic functional groups). In the above formula R1 is any alkane from C1-C25 or H with number of double bonds from 0-5, aromatic moieties, polysiloxane, or mixtures thereof. Furthermore, R1 can be an alkoxylated fatty alcohol with any alkoxy carbon-length, number of alkoxy groups and C1-C25 alkyl chain length. R1 can also be a liquid crystalline moiety that can render the polymer thermotropic liquid crystalline properties, or the alkanes selected can result in side-chain melting. R2 is H or CH3; R3 is alkyl alcohol C1-25 or an alkylene oxide with any number of double bonds, or R3 may be absent such that the C═O bond is (via the C-atom) directly connected to R4. R4 can be: —NH2, NHR1, —NR1R2, —NR1R2R6 (where R6=R1, R2, or —CH2-COOH or its salt), —NH—C(O)—, sulfo betaine, betaine, polyethylene oxide, poly(ethyleneoxide/propylene oxide/butylene oxide) grafts with any end group, H, OH, styrene sulfonate, pyridine, quaternized pyridine, alkyl-substituted pyrrolidone or pyridine, pyridine-N-oxide, imidazolinium halide, imidazolium halide, imidazol, piperidine, —OR1, —OH, —COOH alkali salt, sulfonate, ethoxy sulphate, pyrrolidone, caprolactam, phenyl-R4 or naphthalene-R5 where R4 and R5 are R1, R2, R3, sulfonic acid or its alkali salt or organic counter ion. Any mixture or these R3 groups may be used. Also, glyoxylated cationic polyacrylamides can be used. Typical polymers of choice are those containing the cationic monomer dimethylaminoethyl methacrylate (DMAEMA) or methacrylamidopropyl trimethyl ammonium chloride (MAPTAC). DMAEMA can be found in Gafquat and Gaffix VC-713 polymers from ISP. MAPTAC can be found in BASF's Luviquat PQ11 PN and ISP's Gafquat HS100.

Another group of polymers that can be used are those that contain cationic groups in the main chain or backbone. Included in this group are:

(1) polyalkylene imines such as polyethylene imine, commercially available as Lupasol from BASF. Any molecular weight and any degree of crosslinking of this polymer can be used in the present invention;

(2) ionenes having the general formula set forth as

[N(+)R1R2-A1-N(R5)-X—N(R6)-A2-N(+)R3R4-A3]n-2Z-, as disclosed in U.S. Pat. No. 4,395,541 and U.S. Pat. No. 4,597,962;

(3) adipic acid/dimethyl amino hydroxypropyl diethylene triamine copolymers, such as Cartaretin F-4 and F-23, commercially available from Sandoz;

(4) polymers of the general formula-[N(CH3)2-(CH2)x-NH—(CO)—NH—(CH2)y-N(CH3)2)-(CH2)z-O—(CH2)p]n-, with x, y, z, p=1-12, and n according to the molecular weight requirements. Examples are Polyquaternium 2 (Mirapol A-15), Polyquaternium-17 (Mirapol AD-1), and Polyquaternium-18 (Mirapol AZ-1).

Other polymers include cationic polysiloxanes and cationic polysiloxanes with carbon-based grafts with a net theoretical positive charge or equal to zero (mixture of cationic and anionic functional groups). This includes cationic end-group functionalized silicones (i.e. Polyquaternium-80). Silicones with general structure: —[—Si(R1)(R2)-O-]x-[Si(R3)(R2)-O-]y- where R1 is any alkane from C1-C25 or H with number of double bonds from 0-5, aromatic moieties, polysiloxane grafts, or mixtures thereof. R1 can also be a liquid crystalline moiety that can render the polymer thermotropic liquid crystalline properties, or the alkanes selected can result in side-chain melting. R2 can be H or CH3 and R3 can be —R1-R4, where R4 can be —NH2, —NHR1, —NR1R2, —NR1R2R6 (where R6=R1, R2, or —CH2-COOH or its salt), —NH—C(O)—, —COOH, —COO— alkali salt, any C₁₋₂₅ alcohol, —C(O)—NH2 (amide), —C(O)—N(R2)(R2′)(R2″), sulfo betaine, betaine, polyethylene oxide, poly(ethyleneoxide/propylene oxide/butylene oxide) grafts with any end group, H, —OH, styrene sulfonate, pyridine, quaternized pyridine, alkyl-substituted pyrrolidone or pyridine, pyridine-N-oxide, imidazolinium halide, imidazolium halide, imidazol, piperidine, pyrrolidone, caprolactam, —COOH, —COO— alkali salt, sulfonate, ethoxy sulphate phenyl-R5 or naphthalene-R6 where R5 and R6 are R1, R2, R3, sulfonic acid or its alkali salt or organic counter ion. R3 can also be —(CH2)x-O—CH2-CH(OH)—CH2-N(CH3)2-CH2-COOH and its salts. Any mixture of these R3 groups can be selected. X and y can be varied as long as the theoretical net charge of the polymer is zero (amphoteric) or positive. In addition, polysiloxanes containing up to 5 different types of monomeric units may be used. Examples of suitable polysiloxanes are found in U.S. Pat. Nos. 4,395,541 4,597,962 and U.S. Pat. No. 6,200,554. Another group of polymers that can be used to improve microcapsule/particle deposition are phospholipids that are modified with cationic polysiloxanes. Examples of these polymers are found in U.S. Pat. No. 5,849,313, WO Patent Application 9518096A1 and European Patent EP0737183B1.

Furthermore, copolymers of silicones and polysaccharides and proteins which arecommercially available as CRODASONE brand products.

Another class of polymers include polyethylene oxide-co-propyleneoxide-co-butylene oxide polymers of any ethylene oxide/propylene oxide/butylene oxide ratio with cationic groups resulting in a net theoretical positive charge or equal to zero (amphoteric). The general structure is:

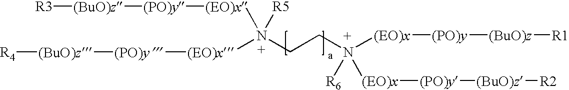

where R1, 2, 3, 4 is —NH2, —N(R)3-X+, R with R being H or any alkyl group. R5, 6 is —CH3 or H. The value for ‘a’ can range from 1-100. Counter ions can be any halide ion or organic counter ion. X, Y, may be any integer, any distribution with an average and a standard deviation and all 12 can be different. Examples of such polymers are the commercially available TETRONIC brand polymers.

Suitable polyheterocyclic (the different molecules appearing in the backbone) polymers include the piperazine-alkylene main chain copolymers disclosed in Ind. Eng. Chem. Fundam., (1986), 25, pp. 120-125, by Isamu Kashiki and Akira Suzuki.

Also suitable for use in the present invention are copolymers containing monomers with a cationic charge in the primary polymer chain, up to 5 different types of monomers may be used. Any co-monomer from the types listed in this specification may also be used. Examples of such polymers are poly diallyl dimethyl ammonium halides (PolyDADMAC) copolymers of DADMAC with vinyl pyrrolidone, acrylamides, imidazoles, imidazolinium halides, etc. These polymers are disclosed in Henkel EP0327927A2 and PCT Patent Application 01/62376A1. Also suitable are Polyquaternium-6 (Merquat 100), Polyquaternium-7 (Merquats S, 550, and 2200), Polyquaternium-22 (Merquats 280 and 295) and Polyquaternium-39 (Merquat Plus 3330), available from Ondeo Nalco.

Polymers containing non-nitrogen cationic monomers of the general type —CH2-C(R1)(R2-R3-R4)-can be used with:

R1 being a —H or C1-C20 hydrocarbon. R2 is a disubstituted benzene ring or an ester, ether, or amide linkage. R3 is a C1-C20 hydrocarbon, preferably C1-C10, more preferably C1-C4. R4 can be a trialkyl phosphonium, dialkyl sulfonium, or a benzopyrilium group, each with a halide counter ion. Alkyl groups for R4 are C1-C20 hydrocarbon, most preferably methyl and t-butyl. These monomers can be copolymerized with up to 5 different types of monomers. Any co-monomer from the types listed in this specification may also be used.

Substantivity of these polymers may be further improved through formulation with cationic, amphoteric and nonionic surfactants and emulsifiers, or by coacervate formation between surfactants and polymers or between different polymers. Combinations of polymeric systems, including those mentioned previously, may be used for this purpose as well as those disclosed in European Patent Application No. EP0672409.

Furthermore, polymerization of the monomers listed above into a block, graft or star (with various arms) polymers can often increase the substantivity toward various surfaces. The monomers in the various blocks, graft and arms can be selected from the various polymer classes listed in this specification and the sources below:

Encyclopedia of Polymers and Thickeners for Cosmetics, Robert Lochhead and William From, in Cosmetics & Toiletries, Vol. 108, May 1993, pp. 95-138;

Modified Starches: Properties & Uses, O. B. Wurzburg, CRC Press, 1986. Specifically, Chapters 3, 8, and 10;

U.S. Pat. Nos. 6,190,678 and 6,200,554; and

PCT Patent Application WO 01/62376A1 assigned to Henkel.

Polymers, or mixtures of the following polymers:

(a) Polymers comprising reaction products between polyamines and (chloromethyl) oxirane or (bromomethyl) oxirane. Polyamines being 2(R1)N—[—R2-N(R1)-]n-R2-N(R1)2, 2HN—R1-NH2, 2HN—R2-N(R1)2 and 1H-Imidazole. Also, the polyamine can be melamine. R1 in the polyamine being H or methyl. R2 being alkylene groups of C1-C20 or phenylene groups. Examples of such polymers are known under the CAS numbers 67953-56-4 and 68797-57-9. The ratio of (chloromethyl) oxirane to polyamine in the cationic polymer ranges from 0.05-0.95.

(b) Polymers comprising reaction products of alkanedioic acids, polyamines and (chloromethyl) oxirane or (bromomethyl) oxirane. Alkane groups in alkanedioic acids C0-C20. Polyamine structures are as mentioned in (a). Additional reagents for the polymer are dimethyl amine, aziridine and polyalkylene oxide (of any molecular weight but, at least, di-hydroxy terminated; alkylene group being C1-20, preferably C2-4). The polyalkylene oxide polymers that can also be used are the Tetronics series. Examples of polymers mentioned here are known under the CAS numbers 68583-79-9 (additional reagent being dimethyl amine), 96387-48-3 (additional reagent being urea), and 167678-45-7 (additional reagents being polyethylene oxide and aziridine). These reagents can be used in any ratio.

(c) Polyamido Amine and Polyaminoamide-epichlorohydrin resins, as described by David Devore and Stephen Fisher in Tappi Journal, vol. 76, No. 8, pp. 121-128 (1993). Also referenced herein is “Polyamide-polyamine-epichlorohydrin resins” by W. W. Moyer and R. A. Stagg in Wet-Strength in Paper and Paperboard, Tappi Monograph Series No. 29, Tappi Press (1965), Ch. 3, 33-37.

The preferred cationically charged material comprises reaction products of polyamines and (chloromethyl) oxirane. In particular, reaction products of 1H-imidazole and (chloromethyl) oxirane, known under CAS number 68797-57-9. Also preferred are polymers comprising reaction products of 1,6-hexanediamine, N-(6-aminohexyl) and (chloromethyl) oxirane, known under CAS number 67953-56-4. The preferred weight ratio of the imidazole polymer and the hexanediamine, amino hexyl polymer is from about 5:95 to about 95:5 weight percent and preferably from about 25:75 to about 75:25.

According to the present invention, the encapsulated fragrance is well suited for a variety of applications, including wash-off products. Wash-off products are understood to be those products that are applied for a given period of time and then are removed. These products are common in areas such as laundry products, and include detergents, fabric conditioners, and the like; as well as personal care products which include shampoos, conditioner, hair colors and dyes, hair rinses, body washes, soaps and the like.

Microcapsules containing an active material, preferably perfume, suitable for use in the present compositions are described in detail in, e.g., U.S. Pat. Nos. 3,888,689; 4,520,142; 5,126,061 and 5,591,146.

The fragrances suitable for use in this invention include without limitation, any combination of fragrance, essential oil, plant extract or mixture thereof that is compatible with, and capable of being encapsulated by a polymer.

Many types of fragrances can be employed in the present invention, the only limitation being the compatibility and ability to be encapsulated by the polymer being employed, and compatibility with the encapsulation process used. Suitable fragrances include but are not limited to fruits such as almond, apple, cherry, grape, pear, pineapple, orange, strawberry, raspberry; musk, flower scents such as lavender-like, rose-like, iris-like, and carnation-like. Other pleasant scents include herbal scents such as rosemary, thyme, and sage; and woodland scents derived from pine, spruce and other forest smells. Fragrances may also be derived from various oils, such as essential oils, or from plant material such as peppermint, spearmint and the like. Other familiar and popular smells can also be employed such as baby powder, popcorn, pizza, cotton candy and the like in the present invention.

A list of suitable fragrances is provided in U.S. Pat. Nos. 4,534,891, 5,112,688 and 5,145,842. Another source of suitable fragrances is found in Perfumes Cosmetics and Soaps, Second Edition, edited by W. A. Poucher, 1959. Among the fragrances provided in this treatise are acacia, cassie, chypre, cylamen, fern, gardenia, hawthorn, heliotrope, honeysuckle, hyacinth, jasmine, lilac, lily, magnolia, mimosa, narcissus, freshly-cut hay, orange blossom, orchids, reseda, sweet pea, trefle, tuberose, vanilla, violet, wallflower, and the like. Those with skill in the art appreciate that fragrance formulations are frequently complex mixtures of many fragrance ingredients. A perfumer commonly has several thousand fragrance chemicals to work from. Those with skill in the art appreciate that the present invention may contain a single ingredient, but it is much more likely that the present invention will comprise at least eight or more fragrance chemicals, more likely to contain twelve or more and often twenty or more fragrance chemicals. The present invention also contemplates the use of complex fragrance formulations containing fifty or more fragrance chemicals, seventy five or more or even a hundred or more fragrance chemicals in a fragrance formulation.

The level of fragrance in the microcapsule product varies from about 0.1 to about 95 weight percent (%), preferably from about 1 to about 80 weight % and most preferably from about 5 to about 60 weight %. In addition to the fragrance, other material can be used in conjunction with the fragrance and are understood to be included.

As used herein olfactory effective amount is understood to mean the amount of compound in perfume compositions the individual component will contribute to its particular olfactory characteristics, but the olfactory effect of the fragrance composition will be the sum of the effects of each of the fragrance ingredients. Thus the compounds of the invention can be used to alter the aroma characteristics of the perfume composition by modifying the olfactory reaction contributed by another ingredient in the composition. The amount will vary depending on many factors including other ingredients, their relative amounts and the effect that is desired.

As noted above, the fragrance may also be combined with a variety of solvents which serve to increase the compatibility of the various material, increase the overall hydrophobicity of the blend, influence the vapor pressure of the material, or serve to structure the blend. Solvents performing these functions are well known in the art and include mineral oils, triglyceride oils, silicone oils, fats, waxes, fatty alcohols, diisodecyl adipate and diethyl phthalate among others.

A common feature of many encapsulation processes is that they require the fragrance material to be encapsulated to be dispersed in aqueous solutions of polymers, pre-condensates, surfactants, and the like prior to formation of the capsule walls. Therefore, material having low solubility in water, such as highly hydrophobic material are preferred, as they will tend to remain in the dispersed perfume phase and partition only slightly into the aqueous solution. Fragrance material with Clog P values greater than 1, preferably greater than 3, and most preferably greater than 5 will thus result in micro-capsules that contain cores most similar to the original composition, and will have less possibility of reacting with material that form the capsule shell.

One object of the present invention is to deposit capsules containing fragrance cores on desired substrates such as cloth, hair, and skin during washing and rinsing processes. Further, it is desired that, once deposited, the capsules release the encapsulated fragrance either by diffusion through the capsule wall, via small cracks or imperfections in the capsule wall caused by drying, physical, or mechanical means, or by large-scale rupture of the capsule wall. In each of these cases, the volatility of the encapsulated perfume material is critical to both the speed and duration of release, which in turn control consumer perception. Thus, fragrance chemicals which have higher volatility as evidenced by normal boiling points of less than 250° C., preferably less than about 225° C. are preferred in cases where quick release and impact of fragrance is desired. Conversely, fragrance chemicals that have lower volatility (boiling points greater than 225° C.) are preferred when a longer duration of aroma is desired. Of course, fragrance chemicals having varying volatility may be combined in any proportions to achieve the desired speed and duration of perception.

The present active material compositions may further comprise one or more malodour counteractant at a level preferably less than about 70 weight %, more preferably less than about 50 weight % of the composition. The malodour counteractant composition serves to reduce or remove malodor from the surfaces or objects being treated with the present compositions. The malodour counteractant composition is preferably selected from uncomplexed cyclodextrin, odor blockers, reactive aldehydes, flavanoids, zeolites, activated carbon, and mixtures thereof. Compositions herein that comprise odor control agents can be used in methods to reduce or remove malodor from surfaces treated with the compositions.

Specific examples of malodour counteractant components useful in aminoplast microencapsulate used in the composition and process of our invention are as follows:

### Malodour Counteractant Component Group I:

1-cyclohexylethan-1-yl butyrate;
1-cyclohexylethan-1-yl acetate;
1-cyclohexylethan-1-ol;
1-(4′-methylethyl)cyclohexylethan-1-yl propionate; and
2′-hydroxy-1′-ethyl(2-phenoxy)acetate each of which compound is marketed under the trademark VEILEX by International Flavors & Fragrances Inc., New York, N.Y., U.S.A. Malodour Counteractant Component Group II, as disclosed in U.S. Pat. No. 6,379,658:

β-naphthyl methyl ether;

β-naphthyl ketone;

benzyl acetone;
mixture of hexahydro-4,7-methanoinden-5-yl propionate and hexahydro-4,7-methanoinden-6-yl propionate;
4-(2,6,6-trimethyl-2-cyclohexen-1-yl)-3-methyl-3-buten-2-one;
3,7-dimethyl-2,6-nonadien-1-nitrile;
dodecahydro-3a,6,6,9a-tetramethylnaphtho(2,1-b)furan;
ethylene glycol cyclic ester of n-dodecanedioic acid;
1-cyclohexadecen-6-one;
1-cycloheptadecen-10-one; and corn mint oil.

In addition to the fragrance material in the present invention contemplates the incorporation of solvent material into the microcapsule product. The solvent material is a hydrophobic material that are miscible in the fragrance material used in the present invention. The solvent material serves to increase the compatibility of various active material, increase the overall hydrophobicity of the blend, influence the vapor pressure of active material, or serve to structure the blend. Suitable solvents are those having reasonable affinity for the fragrance chemicals and a ClogP greater than 2.5, preferably greater than 3.5 and most preferably greater that 5.5. Suitable solvent material include, but are not limited to triglyceride oil, mono and diglycerides, mineral oil, silicone oil, diethyl phthalate, polyalpha olefins, castor oil and isopropyl myristate. In a preferred embodiment the solvent material are combined with fragrance material that have ClogP values as set forth above. It should be noted that selecting a solvent and fragrance with high affinity for each other will result in the most pronounced improvement in stability. Appropriate solvents may be selected from the following non-limiting list:

Mono-, di- and tri-esters, and mixtures thereof, of fatty acids and glycerine. The fatty acid chain can range from C4-C26. Also, the fatty acid chain can have any level of unsaturation. For instance capric/caprylic triglyceride known as Neobee M5 (Stepan Corporation). Other suitable examples are the Capmul series by Abitec Corporation. For instance, Capmul MCM.

Isopropyl myristate
Fatty acid esters of polyglycerol oligomers:

R2CO—[OCH2-CH(OCOR1)-CH2O-]n, where R1 and R2 can be H or C4-26 aliphatic chains, or mixtures thereof, and n ranges between 2-50, preferably 2-30.

Nonionic fatty alcohol alkoxylates like the Neodol and Dobanol surfactants by Shell Corporation or the BioSoft surfactants by Stepan or Utensil by BASF. The alkoxy group being ethoxy, propoxy, butoxy or mixtures thereof. In addition, these surfactants can be end-capped with methyl groups in order to increase their hydrophobicity.

Di- and tri-fatty acid chain containing nonionic, anionic and cationic surfactants, and mixtures thereof.

Fatty acid esters of polyethylene glycol, polypropylene glycol, and polybutylene glycol, or mixtures thereof.

Polyalphaolefins such as the ExxonMobil PureSym™ PAO line

Esters such as the ExxonMobil PureSyn™ Esters

Mineral oil

Silicone oils such polydimethyl siloxane and polydimethylcyclosiloxane

Diethyl phthalate

Di-isodecyl adipate.

While no solvent is needed in the core, in one embodiment the level of solvent in the core of the microcapsule product weight percent should be greater than about 20 weight %, preferably greater than about 50 weight % and most preferably less than about 50 weight %. In addition to the solvent it is preferred that higher ClogP fragrance materials are employed. It is preferred that greater than about 25 weight %, preferably greater than 50 weight % and more preferably greater than about 80 weight % of the fragrance chemicals have ClogP values of greater than about 2.0, preferably greater than about 3.0 and most preferably greater than about 3.5. Those with skill in the art will appreciate that many formulations can be created employing various solvents and fragrance chemicals. The use of high ClogP fragrance chemicals will require a lower level of hydrophobic solvent than fragrance chemicals with lower ClogP to achieve similar stability. As those with skill in the art will appreciate, in a highly preferred embodiment high ClogP fragrance chemicals and hydrophobic solvents comprise greater than about 80 weight %, preferably more than about 90 weight % and most preferably greater than 99 weight % of the fragrance composition.

A common feature of many encapsulation processes is that they require the fragrance material to be encapsulated to be dispersed in aqueous solutions of polymers, pre-condensates, surfactants, and the like prior to formation of the microcapsule walls.

In order to provide the highest fragrance impact from the fragrance encapsulated microcapsules deposited on the various substrates referenced above, it is preferred that material with a high odor-activity be used. Material with high odor-activity can be detected by sensory receptors at low concentrations in air, thus providing high fragrance perception from low levels of deposited microcapsules. This property must be balanced with the volatility as described above. Some of the principles mentioned above are disclosed in U.S. Pat. No. 5,112,688.

As described herein, the present invention is well suited for use in a variety of well-known consumer products such as liquid and powder detergent, laundry detergent and fabric softeners, liquid dish detergents, automatic dish detergents, as well as hair shampoos and conditioners, deodorants and anti-perspirants. These products employ surfactant and emulsifying systems that are well known. For example, fabric softener systems are described in U.S. Pat. Nos. 6,335,315, 5,674,832, 5,759,990, 5,877,145, 5,574,179; 5,562,849, 5,545,350, 5,545,340, 5,411,671, 5,403,499, 5,288,417, and 4,767,547, 4,424,134. Liquid dish detergents are described in U.S. Pat. Nos. 6,069,122 and 5,990,065; automatic dish detergent products are described in U.S. Pat. Nos. 6,020,294, 6,017,871, 5,968,881, 5,962,386, 5,939,373, 5,914,307, 5,902,781, 5,705,464, 5,703,034, 5,703,030, 5,679,630, 5,597,936, 5,581,005, 5,559,261, 4,515,705, 5,169,552, and 4,714,562. Liquid laundry detergents which can use the present invention include those systems described in U.S. Pat. Nos. 5,929,022, 5,916,862, 5,731,278, 5,565,145, 5,470,507, 5,466,802, 5,460,752, 5,458,810, 5,458,809, 5,288,431, 5,194,639, 4,968,451, 4,597,898, 4,561,998, 4,550,862, 4,537,707, 4,537,706, 4,515,705, 4,446,042, and 4,318,818. Shampoo and conditioners that can employ the present invention include those described in U.S. Pat. Nos. 6,162,423, 5,968,286, 5,935,561, 5,932,203, 5,837,661, 5,776,443, 5,756,436, 5,661,118, 5,618,523, 5,275,755, 5,085,857, 4,673,568, 4,387,090 and 4,705,681. All of the above mentioned U.S. patents.

All U.S. patents and patent applications cited herein are incorporated by reference as if set forth herein in their entirety.

The following examples are provided as specific embodiments of the present invention. These and additional modifications and improvements of the present invention may also be apparent to those with ordinary skill in the art. The particular combinations of elements described and illustrated herein are intended only to represent only a certain embodiment of the present invention and are not intended to serve as limitations of alternative articles within the spirit and scope of the invention.

### EXAMPLE ONE

This example illustrates the application of present innovation in producing fragrance encapsulate with titanium dioxide. In this example, 20 g of Uvinul® TiO₂ was blended with 190 g of fragrance Fresh Zion in a 16 Oz jar under stirring to form the core material to be encapsulated. The titanium dioxide was from BASF and has a specific density of 4 g/ml and a primary particle size of 21 nm on the average. The fragrance, Fresh Zion, is a commercially available material from International Flavors & Fragrances Inc. In a separate reaction vessel, 34 gram of a copolymer of acryl amide and acrylic acid was first dispersed in 300 ml of water together with 18 gram of a methylated melamine-formaldehyde resin. These two components were allowed to react under acidic conditions for the desired amount of time at room temperature. The core material consisting of fragrance and TiO₂ was then added to polymer solution. The system was then subject to high shear homogenization to promote mixing and formation of capsules by the pre-formed polymer. Curing of the polymeric layer around the fragrance encapsulate was achieved by increasing the temperature to 50-85° C. and maintaining the temperature for desired amount of time. The slurry was then collected for future use.

It is demonstrated that coating of the fragrance oils containing titanium dioxide is easily accomplished using the present invention.

### EXAMPLE TWO

This example illustrates the use of the present invention in producing fragrance encapsulate at reduced fragrance loading. In this experiment, twenty gram of Uvinul® TiO₂ was mixed with 85 g of NEOBEE M5 oil [Stephan Company, Northfield, Ill.] and 105 gram of Fresh Zion fragrance in a 16 Oz jar under stirring to form the core to be encapsulated. This mixture of fragrance oil, solvent and titanium dioxide was encapsulated similarly according to the process in example one.

Other solvent or diluents can be used in place of Neobee M5 and be encapsulated similarly. This will allow the concentration of the fragrance to be altered easily for the desired perfumery effects.

### EXAMPLE THREE

This example illustrates the use of the present invention in producing fragrance encapsulate with another UV pigment zinc oxide. The zinc oxide, Z-COTE® HP 1 was obtained from BASF. In this experiment, twenty gram of Z-COTE HP1 was mixed with 190 gram of fragrance in a 16 Oz jar under stirring. The fragrance oil containing zinc oxide was encapsulated similarly according to the process in example one.

It is demonstrated that the present invention offer great flexibility as far as the use of inorganic particulate is concerned. Other inorganic particulate such silica can similarly be blended with fragrance oils and be encapsulated.

### EXAMPLE FOUR

This example illustrates the improvement in the rheological profile of the encapsulated material containing titanium dioxide in the core. For comparison, a fragrance capsule without any titanium dioxide was prepared according to the process in example one.

The stability of the capsule was examined by visual inspection. A 16 oz jar was filled with capsule slurry prepared with titanium dioxide in the core using the present invention. The capsule slurry without any titanium dioxide was placed in separate 16 oz jar. The samples were left undisturbed on a bench over a period of one month.

Visible creaming or gelling was observed in the jar containing the capsule slurry without titanium dioxide. The capsule slurry became non-pourable. On the contrary, the capsule slurry containing TiO₂ in the core showed no creaming or jelling. Only a trace layer of water was present in the top of the container. The slurry was pourable with slight agitation. It is quite evident that the rheological property of the slurry has been greatly improved by the encapsulation of TiO₂ in the croe. This is achieved because of the encapsulation of the heavier TiO₂ in the core and its function as a densification agent.

The use of organic and polymeric weighing agent to increase the density of flavor or fragrance oil is well documented. But the use of encapsulated TiO₂ as a densification agent for fragrance and flavoring ingredient is not disclosed. The extreme higher density of TiO₂ makes it a very efficient material for engineering fragrance core material with tunable and desirable core properties.

### EXAMPLE FIVE

This example compares the dilution properties of the fragrance encapsulate prepared by the present invention and fragrance capsules without titanium dioxide in the core. A further benefit of the present invention is established.

The experiments were conducted using Turbiscan Lab commericially available from Formulaction Toulousel, France. A 1% aqueous dispersion was used. The cell used in the experiment measured 5 cm in height and 1 cm in diameter. The back scattering intensity of light is measured periodically as a function of cell height. If upward creaming occurs in the system, the concentration of capsules will decrease in the bottom of the sample cell, resulting a decrease in back scatter intensity. At the same time, an increase in scattering intensity will be observed in the top portion of the sample because of the increase in capsule concentration. If sedimentation occurs instead of upward creaming, the back scattering intensity will increase in the bottom part of the sample and decrease in the top part of the sample.

The results obtained are tabulated in the following table:

TABLE ONE

| Back scattering | | | | |
|---|---|---|---|---|
| intensity | Bottom | Middle | Top | Phenomena |
| Capsule without | Decrease | Unchanged | increase | Creaming |
| TiO₂ | | | | |
| Capsule with | Increase | Unchanged | Decrease | light |
| current | | | | sedimentation |
| invention with | | | | |
| TiO₂ | | | | |
| Capsule with | Increase | Unchanged | Decrease | light |
| current | | | | sedimentation |
| invention with | | | | |
| ZnO | | | | |

It is quite evident that there is no creaming observed in the samples prepared with current invention and only slight sedimentation was observed. In practical applications, this can be eliminated by slight mechanical stirring while upward creaming is hard to remove. Furthermore, the core density can be easily tuned so that sedimentation can be completely eliminated by those skilled in the art using the present invention.

### EXAMPLE SIX

This example further illustrates the application and perfumery benefits of fragrance encapsulate prepared by the current invention.

To evaluate the application benefit, we examined the compatibility of the fragrance encapsulate prepared in example one with a fabric softener containing approximately 24% cationic surfactant. When the fragrances encapsulate containing TiO₂ was added to the surfactant product, immediately dispersion was observed with minimum agitation. In a separate experiment, a small amount of the fragranced TiO₂ was added to the surfactant solution, an oily layer was observed initially and coagulated particle was visibly present even with mixing. This clearly demonstrated that encapsulated material is much easier to use the simple mixture of particulate and oily component because the surface properties of the particulate and fragrance mixture had been beneficially modified by the current invention. The perfumery benefit of this product was further examined by conducting laundry experiment using the fabric softener containing the encapsulated fragrance at 1% neat fragrance. Terry towels were used for the washing experiments. The towels are machined dried per experimental protocol. The perfumery benefit was evaluated by panel of 12 judges. The fragrance intensity is rated from a scale ranging from 0 to 96. A numerical value of 5 would suggest the fabric only produces a very weak intensity while a value of 30 indicates the fabric has a strong smell. The obtained result is given in Table 2.

TABLE 2
Tabulation of Sensory Test Results

| | Pre-rubbing | | Post-rubbing | |
|---|---|---|---|---|
| Samples | intensity | Ratio | intensity | Ratio |
| Blank | 4.6 | 0.9 | 3.5 | 1 |
| Neat | 4.3 | 1.0 | 3.5 | 1 |
| fragrance/No | | | | |
| encapsulation | | | | |
| Encapsulated | 8.9 | 2.1 | 15.2 | 4.3 |
| fragrance | | | | |
| containing TiO₂ | | | | |

Note:
The ratio is calculated with reference to the neat fragrance.

It is quite apparent that the encapsulated samples prepared with the present invention containing TiO₂ produces much higher fragrance intensity at both the pre-rubbing stage and post-rubbing stage. The increase in fragrance intensity is much more pronounced in the post-rubbing stage. This clearly demonstrates that the fragrance encapsulate prepared with the current invention is able to deliver the fragrance much more effectively and to provide the full consumer benefit of the fragrance.

### EXAMPLE SEVEN

This example presents application and perfumery benefits of fragrance encapsulate prepared by the current invention in another commercial fabric softener.

The application benefit was demonstrated by examining the compatibility of the fragrance encapsulate prepared in example one with a fabric softener containing approximately 9% surfactant the remainder being water. When the fragrances encapsulate containing encapsulated TiO₂ was added to the surfactant product, immediately dispersion was observed with minimum agitation. In a separate experiment, a small amount of the fragranced TiO₂ was added to the surfactant solution, an oily layer was observed initially and coagulated particle was visibly present even with mixing.

It is quite evident that encapsulated material is much easier to use the simple mixture of particulate and oily component because the surface properties of the particulate and fragrance mixture had been beneficially modified by the current invention.

The perfumery benefit of this product was further examined by conducting laundry experiment using the fabric softener containing the encapsulated fragrance at 1% neat fragrance. Terry towels were used for the washing experiments. The towels are air dried per experimental protocol. The perfumery benefit was evaluated by panel of 12 judges. The fragrance intensity is rated from a scale ranging from 0 to 96. A numerical value of 5 would suggest the fabric only produce very weak intensity while a value of 30 indicates the fabric has a strong smell. The obtained result is given in Table 3.

TABLE 3
Tabulation of Sensory Test Results

| | Pre-rubbing | | Post-rubbing | |
|---|---|---|---|---|
| Samples | intensity | Ratio | intensity | Ratio |
| Blank | 2.9 | 0.4 | 2.4 | 0.6 |
| Neat | 6.1 | 1 | 4.0 | 1.0 |
| fragrance/No | | | | |
| encapsulation | | | | |
| Encapsulated | 13.6 | 2.2 | 26.1 | 6.5 |
| fragrance/TiO₂ | | | | |

Note:
The ratio is calculated with reference to the neat fragrance.

It is quite apparent that the encapsulated samples prepared with the present invention containing TiO₂ produces much higher fragrance intensity at both the pre-rubbing stage and post-rubbing stage. The increase in fragrance intensity is much more pronounced in the post-rubbing stage. This clearly demonstrates that the fragrance encapsulate prepared with the current invention is able to deliver the fragrance much more effectively and to provide the full consumer benefit of the fragrance.

### EXAMPLE EIGHT

This example demonstrates that significant fragrance transfer and deposition can be realized with the capsules prepared according to the current invention to enhance the application and perfumery benefits of fragrance encapsulate.

To conduct the experiments, two fragrance capsule slurries, capsule one and capsule two, containing the fragrance and TiO₂ were prepared and blended into a fabric softener containing 9% srufactant at 1% fragrance loading. A control product was also prepared using Neat fragrance at 1% fragrance loading. Bench top laundry experiments were the conducted using these solutions and cloth swatches. The washed cloth swatches are air dried per experimental protocol. The headspaces of an equal mass of fabrics were analyzed before and after stirring with steel ball bearings to rupture intact capsules.

TABLE 5

| | Control | Control | Capsule | Capsule | Capsule | Capsule |
|---|---|---|---|---|---|---|
| | Neat | Neat | one | one | two | two |
| | Unstirred | Stirred | Unstirred | Stirred | Unstirred | Stirred |
| Headspace | 2095 | 4244 | 9189 | 81002 | 5100 | 58137 |
| Ratio Stirred/Unstirred | — | 2 | — | 8.8 | | 11.3 |
| Ratio of capsule/neat FZ | | | 4.3 | 19.1 | 2.4 | 13.7 |

It is quite evident that headspace intensity has been greatly increased by the presence of capsule prepared according to the present invention. The increase in intensity is even more pronounced after the capsules are ruptured. The post-rupture intensity is increased at least 13 fold using the capsules prepared by our invention. The further demonstrates the capability of the existence technology as a multifunction delivery system to convey both the application and consumer benefits of fragrance capsules.

### EXAMPLE NINE

This example presents unique application and perfumery benefits of fragrance encapsulate prepared by the current invention. This benefit is illustrated using two capsule systems prepared with and without the use of current invention.

Capsule slurry, without incorporating the current invention, was prepared using a MieCap 003 fragrance commercially available from IFF. In a reaction vessel, 102 gram of a copolymer of acryl amide and acrylic acid was first dispersed in 300 ml of water together with 54 gram of a methylated melamine-formaldehyde resin. These two components were allowed to react under acidic conditions for the desired amount of time at room temperature. After the pre-polymer reached the desired viscosity, 105 g of MieCap 003 was emulsified into the solution under higher shear using a ULTRA TURRAX higher shear mixer at 24000 rpm for 6 minutes. The capsule formed was about one micron observed with an optical microscope. The capsule slurry was then cured at 90 C using a 2 hour heating cycle. This is sample is refereed as smaller and non-densified capsule and labeled as SEncap-ND and used in subsequent evaluations.

Another capsule slurry was prepared using the identical process for SEncap-ND with a core of 95 g of MieCap 003 and 15 g of Uvinul TiO₂. The observed capsule size is about 1 micron with an optical microscopy. This smaller and densified capsule system is labeled as SEncap-DW-TiO₂ and used in subsequent experiments.

To evaluate the perfumery benefit of these two slurries, a laundry experiments were conducted by incorporating the capsule slurry in a fabric conditioner (FC). This FC contains about 9% surfactant with the remainder being water. The encapsulated MieCap was dosed at 0.5% neat fragrance equivalent. Terry towels were used for the washing experiments. The towels are air dried per experimental protocol. The perfumery benefit was evaluated by panel of 12 judges. The fragrance intensity is rated from a scale ranging from 0 to 96. A numerical value of 5 would suggest the fabric only produce very weak intensity while a value of 30 indicates the fabric has a strong smell. The obtained result is given in Table 6.

TABLE 6
Tabulation of Sensory Test Results of Freshly Prepared
samples

| | | Post- | | |
|---|---|---|---|---|
| | Pre-rubbing | rubbing | Ratio of | Improvement |
| | intensity | intensity | Iₚₒₛₜ/ | factor^{a} (%) |
| Samples | (Iₚᵣₑ) | (Iₚₒₛₜ) | Iₚᵣₑ | in Iₚₒₛₜ |
| Blank | 2.2 | 2.1 | 0.6 | |
| SEncap-ND | 3.2 | 5.4 | 1.7 | |
| SEncap-DW-TiO₂ | 3.7 | 16.7 | 4.5 | 210^{a} |

Note to table:
^{a}The improvement factor is calculated by [I_{post(SEncap-DW-TiO2)} − I_{post(SEncap-ND)}]/I_{post(SEncap-ND)} = (16.7 − 5.4)/5.4

It can be than the densified capsules containing TiO₂ has better pre-rubbing intensity and superior post-rubbing intensity than the non-densified capsules. The post-rubbing intensity of the smaller capsule is three times that of the non-densified capsule, an in improvement of 200%.

The benefits of the smaller and densified capsules was further established by aging the RC solutions containing the capsules at 37° C. for 8 weeks and conducting the laundry experiments. The sensory results are tabulated in table 7.

TABLE 7
Tabulation of Sensory Test Results of Aged samples

| | | Post- | | |
|---|---|---|---|---|
| | Pre-rubbing | rubbing | Ratio of | Improvement |
| | intensity | intensity | Iₚₒₛₜ/ | factor^{a} (%) |
| Samples | (Iₚᵣₑ) | (Iₚₒₛₜ) | Iₚᵣₑ | in Iₚₒₛₜ |
| Blank | 2.4 | 2.5 | 0.6 | |
| SEncap-ND | 4.3 | 6.1 | 1.4 | |
| SEncap-DW-TiO₂ | 8.6 | 20.3 | 2.4 | 230^{a} |

Note to table:
^{a}The improvement factor is calculated by [I_{post(SEncap-DW-TiO2)} − I_{post(SEncap-ND)}]/I_{post(SEncap-ND)} = (20.3 − 6.1)/6.1

It is quite apparent that the superior benefit of the densified capsule was retained even when the sample was aged for 8 weeks at 37° C. This suggests that the current invention can provide robust fragrance delivery systems that can provide long-lasting consume benefits.

### EXAMPLE TEN

This example demonstrates the excellent performance of the densified capsule made possible by the current invention in a predominately anionic liquid detergent solution.

To conduct the experiments, the two capsule slurries prepared in Example 10 were incorporated into a liquid detergent product. The detergent contained about 25% surfactants that contained mainly anionic sulfonate. The capsule was mixed into the liquid detergent solution to give a fragrance loading of 0.5% neat equivalent. Laundry and sensory experiments were conducted according the experimental protocols as outlined in Example Five except that the towels were machined dried in these experiments.

The results are presented in Table 8

TABLE 8
Sensory Results in Liquid Detergent

| | | Post- | | |
|---|---|---|---|---|
| | Pre-rubbing | rubbing | Ratio of | Improvement |
| | intensity | intensity | Iₚₒₛₜ/ | factor^{a} (%) |
| Samples | (Iₚᵣₑ) | (Iₚₒₛₜ) | Iₚᵣₑ | in Iₚₒₛₜ |
| Blank | 2.1 | 2.2 | 0.6 | |
| SEncap-ND | 4.6 | 5.0 | 1.10 | |
| SEncap-DW-TiO₂ | 12.7 | 14.0 | 1.10 | 180^{a} |

Note to table:
^{a}The improvement factor is calculated by [I_{post(SEncap-DW-TiO2)} − I_{post(SEncap-ND)}]/I_{post(SEncap-ND)} = (14 − 5.0)/5.0

It can be seen that the smaller capsules and densified capsules containing TiO₂ has superior pre-rubbing and post-rubbing intensity than the smaller capsule without being densified with TiO₂. Both the pre-rubbing and post-rubbing intensity of the smaller capsule is almost three times that of the non-densified capsule, an improvement of close to 200%.

### EXAMPLE ELEVEN

This example demonstrates the excellent deposition of the densified capsule in a liquid detergent solution.

To determine the amount of deposition, the fragrance capsule was dispersed into the detergent solution and laundry experiment was conducted using Terry Towel. After the towel is dried, a piece of fabric cloth (11 cm×14 cm) was cut from the whole towel, weighed, and placed in an 8 oz. jar. Methanol (100 ml) was then added and the container was shaken at 40 min at 220 rpm on table shaker.

Next the jar is placed in a bath sonicator and sonicated for 10 min. The extracted fragrance solution is the analyzed by GC. The results are tabulated in Table 9.

TABLE 9
Deposition Results in Liquid Detergent Solution

| | Deposition, | Improvement |
|---|---|---|
| Samples | (%) | factor^{a} (%) |
| SEncap-ND | 3.0 | |
| SEncap-DW-TiO₂ | 23.0 | 770 |

The improvement factor is calculated by [Deposition_{(SEncap-DW-TiO2)} − Deposition_{(SEncap-ND)}]/Deposition_{(SEncap-ND)} = (23.0 − 3.0)/3.0

It can be seen that the amount of fragrance deposition was dramatically improved in the capsules densified with TiO₂. The amount of deposition is improved by a factor of 800 when the fragrance is densified. This can lead significantly improved consumer benefits.

### EXAMPLE TWELVE

This example illustrates the excellent deposition of the densified capsule on hair fibers. It is commonly know that the deposition of fragrance is extremely low in this type of application.

To determine the amount of deposition, capsules was dispersed in a model body wash base that contains 7% of anionic surfactant solution (sodium lauryl ether sulfate STANDAPOL ES-3®, 28% solution, Cognis Corporation, Cincinnati, Ohio) and 5% amphoteric surfactant(cocamidopropyl betaine, VELVETEX BK-35®, Cognis Corporation, Cincinnati, Ohio). The formulated product was then used to wash hair swatches. Two swatches were used in each wash. All swatches were pre-washed with unfragranced body wash and dried completely. The swatches were wetted under stream of tap water and directly dosed with 0.5 g of product. The hair swathes were lathered between palms of two hands for 30 seconds and rinsed under tap water set to 100 F and 3 L/min flow rate. They were then placed on paper towels place another one on the top and pressed to squeeze excess of water, and let air dry. The waxy part holding the hair together was then removed and the weigh of the swatch was recorded. The two swatches were then placed into separate 6 oz jars. The amount of fragrance deposited is then extracted with 75 ml methanol by shaking for 45 min at about 220 rpm and analyzed by GC. The results are tabulated in Table 11 along with a smaller capsule without densification.

TABLE 11
Deposition Results in Hair Fiber

| | Deposition, | Improvement |
|---|---|---|
| Samples | (%) | factor^{a} (%) |
| SEncap-ND | 2 | |
| SEncap-DW-TiO₂ | 6 | 200 |

The improvement factor is calculated by [Deposition_{(SEncap-DW-TiO2)} − Deposition_{(SEncap-ND)}]/Deposition_{(SEncap-ND)} = (6.0 − 2.0)/2.0

It can be seen that the amount of fragrance deposition was dramatically improved in the capsules densified with TiO₂. The amount of deposition is improved by a factor of 200 when the fragrance is densified. This result is quite remarkable considering the fact that no deposition polymer is used our experiments.

We claim:

1. A capsule particle comprising a combination of an active ingredient, a densifying agent resident largely in the core, and an encapsulating polymeric material.

2. The capsule particle of claim 1 wherein the densifying agent has a density of greater than 1.

3. The capsule particle of claim 1 wherein the densifying agent is selected from a titanium dioxide (TiO₂), zinc oxide (ZnO), Fe₂O₃, CO₂O₃, CoO, NiO, AgO, CuO, Zicornium (ZrO₂), silica, silver (Ag), zinc (Zn), iron (Fe), cobalt (Co), Nickel (Ni), copper (Cu), brominated vegetable oil (BVO), sucrose acetate isobutyrate and mixtures thereof.

4. The capsule particle of claim 1 wherein the densifying agent is present up to about 90% in the capsule particle.

5. The capsule particle of claim 1 wherein the densifying agent is present up to about 50%.

6. The capsule particle of claim 1 wherein the densifying agent is present up to about 20%.

7. The capsule particle of claim 1 wherein the encapsulating polymeric material is a crosslinked network of polymers comprising a melamine-formaldehyde: acrylamide-acrylic acid copolymer wherein the mole ratio is in the range of from about 9:1 to about 1:9.

8. The capsule particle of claim 7 wherein the molar ratio of melamine-formaldehyde: acrylamide-acrylic acid copolymer is in the range of from about 5:1 to about 1:5.

9. The capsule particle of claim 8 wherein the molar ratio of melamine-formaldehyde: acrylamide-acrylic acid copolymer is in the range of from about 2:1 to about 1:2.

10. The capsule particle of claim 1 wherein the encapsulating polymeric material is selected from the group consisting of a polyurethane polymer, a polyurea polymer, a mixture of a polyurethane polymer and a polyurea polymer mixture.

11. The capsule particle of claim 1 wherein the encapsulating polymer is an amine-containing polymer selected from the group consisting of polyvinyl amines, polyvinyl formamides, polyallyl amines, proteins such as gelatin, zein, albumen, polysaccharides and mixtures thereof.

12. The capsule particle of claim 1 wherein the encapsulating polymeric material is an inorganic polymer containing the element silica, titanium, alumina, zinc or a mixture thereof.

13. The capsule particle of claim 1 wherein the capsule particle is further coated with a polymer coating material.

14. The capsule particle of claim 13 wherein the polymer coating material is cationically charged.

15. The capsule particle of claim 13 wherein the polymer coating material is selected from the group consisting of polysaccharides, cationically modified starch, cationically modified guar, polysiloxanes, poly diallyl dimethyl ammonium halides, copolymers of poly diallyl dimethyl ammonium chloride and vinyl pyrrolidone, acrylamides, imidazoles, imidazolinium halides, imidazolium halides, poly vinyl amine, copolymers of poly vinyl amine and N-vinyl formamide, and mixtures thereof.

16. The capsule particle of claim 1 wherein the active ingredient is selected from the group consisting of fragrances, flavoring agents, fungicide, brighteners, antistatic agents, wrinkle control agents, fabric softener actives, hard surface cleaning actives, skin and/or hair conditioning agents, antimicrobial actives, UV protection agents, insect repellents, animal/vermin repellents, flame retardants, and mixtures thereof.

17. A process for preparing a densified capsule particle comprising:
emulsifying a active ingredient, a densifying agent, and an aqueous phase polymer solution comprising an encapsulating polymer to form an emulsion;
curing the emulsion; and
providing a capsule particle with a diameter from about 0.1 to about 2000 microns.

18. The process of claim 17 wherein the density agent has a density of greater than 1.

19. The process of claim 17 wherein the densifying agent is selected from the group consisting of titanium dioxide (TiO₂), zinc oxide (ZnO), Fe₂O₃, CO₂O₃, CoO, NiO, AgO, CuO, Zicornium (ZrO₂), silica, silver (Ag), zinc (Zn), iron (Fe), cobalt (Co), Nickel (Ni), copper (Cu), brominated vegetable oil (BVO), sucrose acetate isobutyrate and mixtures thereof.

20. The process of claim 17 wherein the densifying agent is present up to about 90% in the capsule particle.

21. The process of claim 17 wherein the densifying agent is present up to about 50%.

22. The process of claim 17 wherein the densifying agent is present up to about 30%.

23. The process of claim 17 wherein the oil phase is present from about 1 to about 60% and the aqueous polymer solution is present from about 1 to about 20%.

24. The process of claim 17 wherein the oil phase is present from about 10% to about 45% and the aqueous polymer solution is present from about 2 to about 10%.

25. The process of claim 17 wherein the emulsion is cured at a temperature from about 60 to about 180° C.

26. The process of claim 17 wherein the encapsulating polymer is a crosslinked network of polymers selected from the group consisting of a vinyl polymer, an acrylate polymer, an acrylate acrylamide copolymer, melamine-formaldehyde polymer, urea-formaldehyde polymer, and mixtures thereof to form a polymer encapsulated fragrance.

27. The process of claim 26 wherein the crosslinked network of polymers comprises a melamine-formaldehyde: acrylamide-acrylic acid copolymer wherein the mole ratio is in the range of from about 9:1 to about 1:9.

28. The process of claim 27 wherein the molar ratio of melamine-formaldehyde: acrylamide-acrylic acid copolymer is in the range of from about 5:1 to about 1:5.

29. The process of claim 27 wherein the molar ratio of melamine-formaldehyde: acrylamide-acrylic acid copolymer is in the range of from about 2:1 to about 1:2.

30. The capsule particle of claim 17 wherein the encapsulating polymeric material is selected from the group consisting of polyurethane polymer, a polyurea polymer, a mixture of polyurethane polymer and a polyurea polymer mixture.

31. The capsule particle of claim 17 wherein the encapsulating polymeric material is an inorganic polymer containing the element silica, titanium, alumina, zinc, and mixtures thereof.

32. The process of claim 17 wherein comprising the additional step of coating the capsule particle with a polymer coating material.

33. The process of claim 32 wherein the polymer coating is cationically charged.

34. The process of claim 17 wherein the active ingredient selected from the group consisting of fragrances, flavoring agents, fungicide, brighteners, antistatic agents, wrinkle control agents, fabric softener actives, hard surface cleaning actives, skin and/or hair conditioning agents, antimicrobial actives, UV protection agents, insect repellents, animal/vermin repellents, flame retardants, and mixtures thereof.

35. A consumer product comprising the densified capsule particle prepared according to the process of claim 17.

36. The consumer product of claim 35 wherein the consumer product is selected from the group consisting rinse conditioners, detergents, industrial and institutional cleaners, personal care, shampoo, conditioner, body wash, anti-perspirant, deodorant, sunscreens, lotions and hair styling products.
